(19) United States
(12) Patent Application Publication
(10) Pub. No.: US 2022/0331492 A1
(43) Pub. Date: Oct. 20, 2022

(54) COMPOSITION AND METHOD FOR CONTROLLED DRUG RELEASE FROM A TISSUE

(71) Applicant: Bard Shannon Limited, Humacao, PR (US)

(72) Inventors: Guy Tomer, Modiin (IL); Aurelie Benaddi, Netanya (IL); Moriah Anouchi, Zikhron Yakov (IL); Amir Hadid, Binyamina (IL)

(73) Assignee: Bard Shannon Limited, Humacao, PR (US)

(21) Appl. No.: 17/741,358

(22) Filed: May 10, 2022

Related U.S. Application Data
(63) Continuation of application No. 16/650,956, filed on Mar. 26, 2020.
(60) Provisional application No. 62/565,147, filed on Sep. 29, 2017.

(51) Int. Cl. A61L 27/52; A61K 9/16; A61K 31/496; A61K 31/7036; A61L 27/22; A61L 27/48; A61L 27/54

(52) U.S. Cl. CPC A61L 27/52; A61K 9/1658; A61K 31/496; A61K 31/7036; A61L 27/222; A61L 27/48; A61L 27/54; A61L 2300/406; A61L 2300/622

(57) ABSTRACT

A composition, comprising a hydrogel matrix and microparticles within said matrix, said matrix comprising a cross-linkable protein and a cross-linking agent, wherein said cross-linking agent is able to cross-link said cross-linkable protein, wherein said microparticles comprise a drug.

### RELATED APPLICATIONS

This application is a continuation of U.S. patent application Ser. No. 16/650,956, filed Mar. 26, 2020, titled “COMPOSITION AND METHOD FOR CONTROLLED DRUG RELEASE FROM A TISSUE,” which is a national stage filing under 35 U.S.C. § 371 of International Patent Application Ser. No. PCT/IL2018/050383, filed Mar. 29, 2018, titled “COMPOSITION AND METHOD FOR CONTROLLED DRUG RELEASE FROM A TISSUE,” which claims the benefit of U.S. Provisional Application Ser. No. 62/565,147, filed Sep. 29, 2017, titled “COMPOSITION AND METHOD FOR CONTROLLED DRUG RELEASE FROM A TISSUE.” Each of these applications is incorporated herein by reference in its entirety.

### FIELD OF THE INVENTION

The present invention is of a composition and method for controlled drug release on a target tissue, and in particular for such a composition and method for controlled antibiotic release to an infected tissue or tissue which is prone to infection.

### BACKGROUND OF THE INVENTION

In several medical situations, infections arise which are difficult to treat using a systemic antibiotic administration. The concentration at the infected site is too low as a result of poor blood supply, so that the drug in the circulation cannot reach the site efficiently. In this case increasing the systemic exposure may increase the local concentration at the site to the desired values; however this approach is prohibited due to the systemic toxicity of the antibiotics, which in most cases is the limiting factor. A scenario of poor blood supply at infected sites can arise as a result of tissue trauma or an ensuing inflammation or necrosis. Another medical situation that leads to poor treatment outcomes of systemic antibiotic intervention is the development of a biofilm. Biofilms are a dense layer of bacteria, which is made of or encapsulated with a polysaccharide secretion called glycocalyx. This layer comprises a barrier that shields the bacteria from the effect of antibiotics, thus necessitates the use of higher and higher concentration of these drugs. In addition to the reduced efficacy and danger of toxicity associated with the use of antibiotics to treat such infected sites, systemic treatment with antibiotics is the number one cause in the development of bacterial resistance, and this is an emerging global healthcare concern.

Examples of medical conditions in which local antibiotic therapy is preferred over systemic exposure include but are not limited to osteomyelitis, bone fractures treated with metal rods, plates or external fixators. The risk is especially high with open fractures, total joint replacement, vascular bypass surgery with the use of artificial graft material, general surgical procedures such as hernia repair and various procedures performed on the uterus and bladder and in chronic infected wounds such as ulcers. In these applications the released antibiotics is used to eradicate existing infection and in others prophylactically.

Diabetic foot ulcers that are associated with osteomyelitis impose a substantial burden on public and private payers in the United States, doubling care costs per patient compared with diabetic patients without foot ulcers. Ulcer care adds around US $9 billion to $13 billion to the direct yearly costs associated with diabetes itself (http://www.medscape.com/viewarticle/821908), and are due to increased hospitalization costs as a result of foot amputations. The incidence of nontraumatic lower extremity amputations (LEAs) has been reported to be at least 15 times greater in those with diabetes than with any other concomitant medical illness. LEA is less common but is an extreme complication associated with diabetes and foot ulcer. In the U.S., nearly 80,000 LEAs are performed on diabetics each year in 2005, the overall rate of hospital discharge for new LEA was about 4.3 per 1,000 people with diabetes compared with a rate of about 0.3 per 1.000 in the general population. The annual mortality rate for diabetic patients who have an incident diabetic foot ulcer is about 11%; for those with an incident lower extremity amputation, about 22% (Margolis D J et al).

Antibiotics are most commonly incorporated into polymethylmethacrylate (PMMA) cement, which can then be formed into beads, molded to fit a bone defect. The problem with PMMA beads is that they emit considerable heat upon polymerization which by itself might cause thermal damage to the antibiotic drug. In addition the beads are not degradable, and the patient must undergo a second surgery to remove them before given a bone implant. In addition, PMMA beads must be mixed with the desired antibiotics in the surgical arena prior to the surgery. This is tedious and time consuming, and also incorporates a risk of improper mixing resulting in exposure to hyper or sub quantities of the antibiotic drug. To circumvent some of the problems outlined above with PMMA beads degradable bone substitutes have been developed. For example, Cerament Bone Void Filler (Bonesupport, Sweden) is a degradable synthetic calcium sulfate bone substitute. In Europe it is sold also as a pre-mixed version with antibiotics under the name Cerament G (contains gentamycin) and Cerament V (contains vancomycin). According to the manufacturer's brochure, the antibiotics elutes with a high initial peak that remains above the MIC for 28 days for Staphylococcus aureus and Pseudomonas aeruginosa. There is no evidence however of controlled release, the embedded antibiotic is released by diffusion according to concentration gradient, this is less desired as the release profile might be influenced by the chemical and physical conditions at the site of implantation which are unique to each patient resulting in increased variability of the outcomes, reduced efficacy and predictability of the treatment outcome U.S. Pat. No. 9,180,137 B2 discusses calcium sulfate based bone cement with the addition of antibiotics.

Other attempts to deliver antibiotics to an infected site included premixing the drug with fibrin glue. The intention is that the fibrin glue serves as a delivery vehicle bringing the drug into the infected site, and retaining it there as the beads are entrapped in the hydrogel. Kara et al mixed antibiotics (moxifloxacin, lomefloxacin, vancomycin, and ceftazidime) with fibrin glue and measured the radius of inhibition on petri plates covered with various bacterial strains The drugs eluted up to 72 hours, and the elution did rot show zero order kinetics but rather according to concentration gradient—most of the drugs were released during the first 24 hours. Tredwell et al mixed cefazolin with fibrin glue cefazolin was released in a controlled manner over 2 days, with most being released during the first day. Cashman et al blended the antibiotics cefazolin, fusidic acid or 5-fluorouracil into Vitagel tissue sealant (fibrin glue with the addition of microfibrillar collagen). The drugs were released in a controlled manner over 2-4 days. The above examples demonstrate that when drug are embedded in fibrin glue, it diffuses out of the matrix according to its concentration gradient, namely by a first order kinetics. This is because the drug is much smaller than the pores of fibrin glue and most other hydrogel, so it is free to diffuse out.

Penn-Barwell et al tested a bioabsorbable phospholipid gel, designated DFA-02 containing 1.88% vancomycin and 1.68% gentamicin by weight in an open fracture model in rats contaminated with Staphylococcus aureus. The outcome was better than PMMA beads containing the same drugs. However, concentrations of both antibiotics dropped rapidly within the first 48 hours.

Local delivery of antibiotics is used to treat soft tissue infections as well For instance, gentamicin-impregnated collagen sponges have been tested as a bioabsorbable vehicle (CollaRx, Innocoll, Gallowston, Ireland). However, in a non-orthopedic clinical randomized controlled trial the sponge group had a higher rate of surgical site infection and it was speculated that the antibiotics eluted faster than the sponge degraded, leaving foreign material in the wounds without antibiotics (Bennett-Guerrero et al).

For an efficient local antibiotic therapy it would be better to achieve tight control over the release. A release with a zero order kinetics is the gold standard of drug release. One way to achieve controlled release is to encapsulate the drug in microparticles (MPs) made from degradable hydrophobic materials, such as PLGA (polylactic glycolic acid) or PCL (polycaprolactone). The hydrophilic drug has to pass the hydrophobic matrix on its way out of the MP, which retards its solubility and diffusion resulting in zero order kinetics release, i.e. a constant release rate over time.

Setterstrom et al (U.S. Pat. No. 6,410,056) developed PLGA MPs containing ampicillin or cefazolin to treat various infection models in rats including rat soft tissue wound infection model and rat fraction fixation model inoculated with Staphylococcus aureus, Streptococcus pyogenes or E. coli.

The challenge with using beads to treat local infections is how to retain them in the target site without their migration out of there. Due to their small size, the beads will tend to migrate away, thus the effective amount of antibiotic drug will be reduced. One approach would be for the surgeon to compress the beads into a compact mass using a surgical tool. For example. Garvin et al teach using gentamycin loaded PLGA microspheres to treat osteomyelitis in a canine model. However, the microspheres were compressed prior to implantation to a 5×15 millimeters rectangular shape implant, thus preventing migration of individual MPs away from the defect site. A drawback of this approach is that the compression reduces the effective surface area of the MPs, and by such the release profile of the drug is affected. In addition, compressing the beads into a space or a cavity is time consuming, and the beads are limited in their ability to penetrate narrow crevices, for example as those found in bone, especially with large particles.

Treating soft tissue infections has always been a challenge because of the inaccessibility of orally or parenterally given antibiotic drugs to the infected site or the toxicity associated with administering a high dose of antibiotics required to treat the infections. Infected diabetic foot ulcers (DFI) are a good example of soft tissue infections that are difficult to treat effectively, and the current therapies fail to provide an adequate means of long term treatment. This is partly due to the chronic nature of the wound and the persistence of the infection which is exacerbated by factors such as impaired wound healing in diabetic patients and the geometrical dimensions of the ulcers, which in many cases are deep and tunneled. The latter makes currently used treatments such as gauzes, bandages and dressings less efficient as their contact area with the wound is limited to its upper external part.

Edwards et al evaluated several randomized controlled trials (RCTs) for debridement of diabetic foot ulcers (DFU) and concluded that hydrogels are significantly more effective than gauze or standard care in healing diabetic foot ulcers. Certain groups took this approach further and developed injectable hydrogels that are to be injected into the wound depth Marston et al treated patients having chronic DFU with an injectable porcine collagen-derived matrix and reported a 72% reduction in wound size 2 weeks after injection Campittello et al treated 18 patients with tunneled or cavity ulcers with an injectable matrix composed of crosslinked collagen and glycosaminoglycan which forms a gel in the body. According to the authors of the study 89% of the patients showed complete regeneration of the wound.

Antibacterial drugs have been combined in dry matrices as well for treating soft tissue infections. For example. Gentamicin Surgical implant and CollaRx Gentamicin Topical (Innocoll Pharmaceuticals) are products made from a collagen sponge containing gentamicin, indicated for use in surgical site infections and diabetic foot infections (DFI), respectively. A clinical trial on 56 randomized patients with DFI showed some initial good results in patients who were treated with gentamicin impregnated collagen sponge vs placebo sponge vs. without sponge (Lipsky et al, 2012). However a much larger phase 3 study with this sponge (now called COGENZIA) did not achieve statistical significance in improving clinical cure in diabetic foot infections (DFI). A similar product, COLLATAMP G is a lyophilized bovine Type I collagen matrix impregnated with gentamycin is indicated for surgical site infections. The manufacturer's web site shows the drug release profile, and it is clear that there was no controlled release profile of the antibiotics, and that the drug was above the MIC for only 7 days. These limitations often necessitate replacing the products up to several times a day, in order to maintain the drug level above MIC for a prolonged time.

### BRIEF SUMMARY OF THE INVENTION

The background art does not offer a solution to the problem of localized treatment with antibiotics that provides both excellent surface area for drug release, and control over the timing and location of such release. While providing many small beads enables an excellent surface area, the beads tend to migrate away from the location to be treated. A larger implant that retains the beads at the target tissue solves the problem of bead migration but reduces the effective surface area for release and may also result in certain tissues remaining untreated.

The present invention overcomes these drawbacks of the background art by providing a composition and method for localized treatment of a tissue with controlled drug release, which features fixated beads in a hydrogel matrix. The matrix is preferably cross-linked gelatin that is cross-linked in situ, although optionally a different matrix could be used that is also capable of being cross-linked in situ. Without wishing to be limited to a closed list of benefits, the fixation prevents the beads from migrating and retains their surface area while the beads themselves provide a means for controlled drug release. Preferably, the controlled drug release is also sustained.

The composition features encapsulating the drug in polymeric microparticles to achieve zero order release kinetics, and embedding these particles in a hydrogel to allow easy access to the infected site by virtue of their injectability as well as preventing their migration out of the infected site due to hydrogel fixation to the target tissue. This approach of dispersing particulates in hydrogels has been named “plum pudding” by various research groups, and is a considered a subset of composite gels. In addition the hydrogel itself preferably has the following properties to achieve this goal: biocompatible, injectable, degradable (over a period of time longer than the desired release period, not interfering with the activity of the encapsulated drug and promoting cellular growth. In addition the hydrogel preferably shows bioadhesive properties, because the adhesion to tissue is expected to prolong the residence time of the microparticles in the infected site. Another desired property is elasticity, as brittle hydrogel might break down or be subjected to mechanical erosion thus restricting the efficiency of the treatment.

The composition overcomes the drawbacks of previous such “plum pudding” attempts by providing a suitable hydrogel for the polymeric matrix, which preferably comprises cross-linked gelatin. The gelatin is cross-linked in situ, rather than being pre-cross linked, which provides a much better matrix for reasons described in greater detail below. Furthermore the composition itself is preferably bioadhesive, which increases the stability of application to the local tissue.

Other hydrogels have not been shown to be suitable for such a composite approach. For example, fibrin glue is not a suitable delivery vehicle for drugs, as it is degraded quickly in vivo, and will usually be gone within a few days, long before the desired duration of 2-4 weeks of drug release.

US 20110038946 teach the use of injectable polyurethane scaffolds containing PLGA MPs with tobramycin. Polyurethane adhesives are not entirely biocompatible, especially when they are used as injectable, as the components, namely polyisocyanates might diffuse away from the injected mass prior to curing or in case of incomplete curing.

Foox et al teach the use of a gelatin-alginate hydrogel, crosslinked with EDC for antibiotics drug release. The matrix was loaded with antibiotics (clindamycin, ofloxacin, vancomycin). Only clindamycin was found to b inert toward the crosslinking reaction and did not decrease the bonding strength of the bioadhesive This was interpreted to be a result of the EDC crosslinker interacting with carboxylic groups found on ofloxacin and vancomycin, and demonstrates the importance of choosing an inert crosslinker for the hydrogel carrier matrix. 100% of the clindamycin contained in the gel was released after 4 hours, again demonstrating the inability of gelatin hydrogels to retain embedded drugs without some sort of encapsulation in polymeric microparticles.

WO2014196943 teach the use of injectable hydrogel containing vancomycin MPs embedded in poloxamer. Poloxamers are the most widely used reverse thermal gelation polymers, however the maximum duration of drug release from poloxamers gel systems is limited by the influx of water which dilutes the polymer below its critical gelation concentration such that the matrix loses gel-like properties (Hoare et al).

Gelatin has been used as a biomaterial for decades. It has been shown to be safe, degradable, and biocompatible by numerous laboratories around the world and is based on a vast accumulated clinical experience. In contrast to hydrogels made from synthetic polymers such as poloxamers mentioned above or PEG, gelatin has a favorable tissue response and allow cellular in-growth, in part because it contains abundant Arg-Gly-Asp (RGD) sequences which are the cell attachment sites recognized by many integrins. The mechanical properties of gelatin hydrogels can be enhanced by crosslinking, by either physical, chemical or enzymatic means. Of note is enzymatic crosslinking of gelatin, induced by microbial transglutaminase, as described for example in U.S. Pat. Nos. 8,367,388 and 9,017,664, both owned in common with the instant application, both of which are hereby incorporated by reference as if fully set forth herein. This type of crosslinker is safer to use than conventional means of crosslinking, i.e. glutaraldehyde or formaldehyde

Another advantage of crosslinked gelatin hydrogels as carriers for drug delivery is that it is injectable, and its degree of crosslinking can be adjusted to allow custom made degradation rate, as opposed to fibrin glue, which is biocompatible but degrades within a few days, making it unsuitable to many applications where it is required to elute the drug at the infected site for a longer period. Finally, crosslinked gelatin hydrogel possesses favorable mechanical properties that are required to ensure optimal performance at the site of implantation. First, gelatin is inherently bioadhesive, and demonstrates tackiness to various tissues. The bonding strength of gelatin to tissues is due to the functional chemical groups on the tissue surface (e.g. lysines) which can interact with similar chemical groups on the gelatin molecule by virtue of Van der Waals and hydrogen bonds. Crosslinking of gelatin contributes further to the bonding strength to tissues as a result of covalent bonds formed between the above mentioned chemical functional groups. Crosslinking also increases the cohesive bonds between gelatin chains by forming intermolecular covalent bridges. This contributes to the cohesive strength of the matrix and the resulting tensile or compression strength. The combination of adhesive strength and cohesive strength ensures that the hydrogel remains attached at the target infected site for the duration required for delivering the drug. Overall, the above suggests that crosslinked gelatin matrices are ideal for drug delivery applications, when cross-linked in situ.

U.S. Pat. No. 8,138,157 describes antibiotic containing microparticles (for example made of PLGA) in a gel, which is described as being made from Floseal (which contains gelatin). However. Floseal is made from pre-crosslinked gel particles. There is no curing process that forms a gel in situ so the separate gel particles do not coalesce into a continuous gel matrix. Therefore any drug eluting particles present in the formulation might migrate out of the treatment site, unlike the situation with in situ crosslinked gelatin matrix. In addition, Floseal is indicated as a hemostat. There is no evidence that it behaves as a glue or bioadhesive, therefore it is not expected to be suitable to be used in drug delivery applications where the drug carrier matrix is required to adhere to tissue at the target delivery site. Furthermore, this patent does not provide any experimental evidence to indicate the efficacy of this solution

It would be advantageous to combine a wound healing promoting scaffold or matrix with drug eluting properties, e.g. antibiotics to eradicate infected deep tunneled or cavity ulcers. Cerament G or Cerament V elute gentamycin and vancomycin, respectively, but are constitute of bone cement and are therefore not suitable for soft tissue repair. There are several commercial topical wound dressing and gels with anti-microbial activity, based on antiseptic agents (e.g. silver ions, iodine and PHMB) or antibiotics (e.g. bacitracin, mupirocin, retapamulin against gram positive bacteria: neomycin and silver sulfadiazine against gram negative bacteria. These topical agents however do not possess controlled drug release properties and in addition are comprised of synthetic polymers with little to no biologic activity. A systematic review of antimicrobial agents for chronic wounds (diabetic foot ulcers, pressure ulcers, chronic leg ulcers, etc.) concluded that few systemic agents improved outcomes (Lipsky et al, 2014). Therefore an improvement to the application and delivery system would clearly be of benefit in treating such chronic wounds.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. In the drawings:

FIGS. 1A-IC show in vitro release of antibiotic drugs;

FIG. 2 shows release of ciprofloxacin MP embedded in enzymatically crosslinked gelatin matrix;

FIGS. 3A-3C show the anti-microbial activity of crosslinked gelatin hydrogel containing MPs with either gentamycin or vancomycin entrapped within the MPs; and

FIG. 4 shows mechanical testing of enzymatically crosslinked gelatin hydrogels.

FIGS. 5A-5C show the size distribution of three different PLGA beads preparations containing different antibiotic drugs.

### DESCRIPTION OF AT LEAST SOME EMBODIMENTS

The present invention, in at least some embodiments, comprises a crosslinked gelatin hydrogel matrix containing microparticles. The particles contain a drug. The drug is released from the microparticles, for example and without limitation, optionally by diffusion or erosion mechanism. The rate of release is determined primarily by the material from which the microparticle is comprised of, but also by other parameters such as the type of drug, its solubility, the amount of the encapsulated drug. The product is preferably injectable, and undergoes in situ curing, which by the inherent adhesiveness to tissues fixates itself onto the target tissue or anatomically defined space such as cavity or crevice.

The gelatin matrix is degradable, injectable and biocompatible. The gelatin is made preferably from type A porcine skin, but can be made from bovine or fish gelatin as well. The gelatin has preferably a bloom of 100-300, more preferably 250-300, but optionally 100-250.

The gelatin matrix may optionally be crosslinked enzymatically, using transglutaminase, preferably from microbial source, but also optionally using mammalian transglutaminase, e.g. pig liver transglutaminase, Factor Xiii etc.

Optionally the gelatin matrix can be crosslinked using a chemical crosslinker such as glutaraldehyde or EDC.

The microparticles are manufactured using methods known to those skilled n the art. Non limiting examples include single emulsion method, double emulsion method, polymerization (normal or inter-facial), phase separation coacervation, spray drying and solvent extraction (for example see Bansal et al).

Microparticles

The microparticles comprise one or more biocompatible polymers. Non-limiting examples of such biodegradable polymers include aliphatic polymers (e.g. polylactic acid, polyglycolic acid, polycitric acid, polymalic acid, polycaprolactone), polycarbonates (e.g. polyethylene carbonate, polyethylene propylene carbonate) and polyamino acids (e.g. poly-γ-benzyl-L-glutamic acid, poly-L-alanine, poly-γ-methyl-L-glutamic acid) These polymers may be homopolymers, copolymers of 2 or more monomers, or a mixture of polymers. They may also be in the salt form.

Polylactic acid may be represented by the following structural formula:

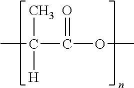

wherein n for example can be any suitable integer between 10 and 250. Polylactic acid can be prepared according to any method known in the state of the art. For example, polylactic acid can be prepared from lactic acid and/or from one or more of D-lactide (i.e. a dilactone, or a cyclic dimer of D-lactic acid), L-lactide (i.e. a dilactone, or a cyclic dimer of L-lactic acid), meso D,L-lactide (i.e. a cyclic dimer of D-, and L-lactic acid), and racemic D,L-lactide (racemic D,L-lactide comprises a 1:1 mixture of D-, and L-lactide). Optionally the polylactic acid polymer comprises poly(L-lactic acid), poly(D,L-lactic acid) or poly(D-lactic acid).

Polyglycolic acid may be represented by the following structural formula:

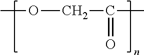

wherein n for example can be any suitable integer between 10 and 250.

Polycaprolactone has the following structure:

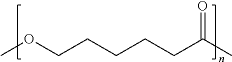

wherein n for example can be any suitable integer between 10 and 250.

Polylactic glycolic acid copolymers have the following unit structure, which is preferably repeated a suitable number of times, for example between 10 and 250 times:

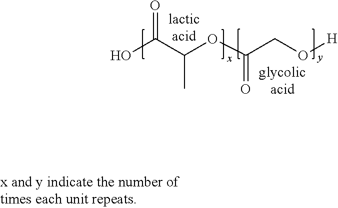

Other non-limiting examples of suitable biocompatible polymers are polystyrene, polyacrylic acid, polymethacrylic acid, polyamides, polyamino acids, silicon polymers, polyurethanes, etc.

Among these polymers, particularly preferred for use in this invention are PLA (polylactic acid), PGA (polyglycolic acid) and PLGA (polylactic glycolic acid) copolymers, optionally in a ratio of lactic acid to glycolic acid n the copolymer from 20:80 to 80:20. Alternatively the polymer is polycaprolactone.

The MPs (microparticles) size optionally ranges from 0.5 to 50 micron.

The MP containing the drug can be dispersed in the gelatin component, the enzyme component or both components of a liquid formulation of crosslinked gelatin. The amount of MP in the final formulation ranges between 1 mg/ml and 50 mg-ml, preferably between 5 mg/ml and 40 mg ml, more preferably between 10 mg ml and 30 mg/ml.

The dispersion of the M's in one or more of the components of the gelatin matrix can be done during the manufacturing of the product or before use in the operating room. In the former case, the MPs are mixed with one of the components, and are stored until use. Since in aqueous environment the encapsulated drug will start to diffuse out of the MPs, and since PLGA is subjected to hydrolysis in aqueous environments, the component containing the MPs is better kept stored at a low temperature, refrigerated or frozen.

Alternatively, the MIPs are kept dry, and are reconstituted with the gelatin or enzyme component just prior to use, in order to keep the MP stable. There are many technical solutions for reconstitution of the dry powder in a liquid formulation and these should be known to those skilled in the art. For example, RISPERDAL® CONSTA® (risperidone) Long-Acting Injection is a combination of extended-release microspheres for injection and diluent for parenteral use. The microspheres are provided dry in a vial, and reconstituted with the supplied diluent prior to injection inside the syringe. Alternatively, the microspheres can be reconstituted with the diluent during the assembly of a single syringe without a need for transfer between a syringe and a vial. An example is Lupron Depot (leuprolide acetate for depot suspension for treatment of prostate cancer) which is supplied as a prefilled dual chamber syringe. This syringe contains powdered microspheres which when mixed with diluent becomes a suspension. The suspension is then administered as a single intramuscular (IM) injection. A third variant is mixing by attaching syringe containing the diluent and a syringe containing the MP particles and passing the content between the syringes for a number of times to make a homogenous suspension. An example for this variant is ELIGARD Injection (leuprolide acetate for injectable suspension).

According to at least some embodiments, drug elution time is adjusted so that the drug elutes from the microparticles over the course of 2 to 6 weeks, preferably 2-5 weeks and more preferably 2-4 weeks, which is the amount of time required to eradicate the bacterial infection as a non-limiting example.

Various optional, non-limiting exemplary embodiments are now described, which may optionally also be combined with each other and/or with any other embodiment or implementation as described herein. According to one embodiment the gelatin sealant containing drug eluting MPs is injected into a cavity formed in bones following debridement of the infected bone tissue in the case of osteomyelitis. After allowing a few minutes for curing, the surgeon makes sure that the formulation has gelled and solidified, before continuing with the surgery or closing the wound. Example 1 below shows that 3 different antibiotic drugs encapsulated in PLGA microparticles are released in a controlled manner following a zero order kinetics after an initial burst release. The PLGA MP containing ciprofloxacin were embedded in an in situ cross-linkable gelatin matrix and the release rate was somewhat slower than MP alone, as a result of the additional diffusion barrier, but nevertheless the release was controlled with a zero order kinetics, and the drug eluted over the course of 2 weeks, which is the amount of time required to eradicate the bacterial infection

Encapsulated Drug

The encapsulated drug may optionally comprise one or more of antibiotics, analgesic, anti inflammatory, or anti-tumor drugs.

For all of the below antibiotics, optionally administration may be as the pharmaceutically acceptable salts or hydrates, and/or combinations of such antibiotics thereof.

Non-limiting examples of antibiotics include: an aminoglycosidic antibiotic a glycopeptide antibiotic, ansamycins, carbacephems, carbapenems, cephalosporins, macrolides, penicillins, polypeptides, quinolones, sulfonamides, tetracyclines, lincosarmides, nitrofurans, nitroimdazoles and mixtures thereof.

Non-limiting examples of aminoglycosidic antibiotics include etimicin, gentamicin, tobramycin, amikacin, netilmicin, dibekacin, kanamycin, arbekacin, sagamicin, isopamicin, sisomicin, neomycin, paromoycin, streptomycin, spectinomycin, micronomicin, astromicin, ribostamycin, pharmaceutically acceptable salts or hydrates, and combinations thereof.

Non-limiting examples of glycopeptide antibiotics include vancomycin, avoparcin, ristocetin, teicoplanin, telavancin, ramoplanin and decaplanin, a derivative of vancomycin, avoparcin, ristocetin, or teicoplanin, pharmaceutically acceptable salts or hydrates, and combinations thereof.

Non-limiting examples of carbacephem antibiotics include loracarbef.

Non-limiting examples of carbapenem antibiotics include ertapenem, meropenem, imipenem cilastatin, panipenem, biapenem and tebipenem.

Non-limiting examples of cephalosporin antibiotics include cefadroxil, cefacetrile, cefalexin, cefaloglycin, cefalonium, cefaloridine, cefalotin, cefapirin, cefatrizine, cefazaflur, cefazedone, cefazolin, cefradine, cefroxadine, ceftezole, cefaclor, cefontcid, cefprozil, cefuroxime, cefamandole, cefuzonam, cefmetazole, cefotetan, cefixime, cefdinir, cefditoren, cefoperazone, cefotaxine, cefpodoxime, ceftazidime, ceftibuten, ceftizoxime, ceftriaxone, cefepime, ceftobiprole and cefoxitin.

Non-limiting examples of macrolide antibiotics include azithromycin, clarithromycin, erythromycin, fidaxomicin, dirithromycin, roxithromycin, troleandomycin, spectinomycin, telithromycin and spiramycin.

Non-limiting examples of penicillin antibiotics include amoxicillin, ampicillin, azlocillin, carbenicillin, cloxacillin, dicloxacillin, flucloxacilline, mezlocillin, meticillin, nafcillin, oxacillin, penicillin, piperacillin, and ticarcillin.

Non-limiting examples of quinolone antibiotics include ciprofloxacin, enoxacin, gatifloxacin, levofloxacin, lomefloxacin, moxifloxacin, norfloxacin, ofloxacin, trovafloxacin, grepafloxacin, sparfloxacin, and temafloxacin.

Non-limiting examples of sulfonamide antibiotics include mafenide, sulfonamidochrysoidine, sulfacetamide, sulfadiazine, sulfamethizole, sulfanilamide, sulfasalazine, sulfisoxazole, trimethoprim-sulfamethoxazole, and cotrimoxazole.

Non-limiting examples of tetracycline antibiotics include doxycycline, minocycline, oxytetracycline, tetracycline.

Non-limiting examples of other suitable antibiotics include aztreonam, a monobactam antibiotic, amphenicol antibiotics such as chloramphenicol and thiamphenicol; ethambutol, fosfomycm, isoniazid, linezolid, mupirocin, platensimycin, pyrazinamide, quinupristin.dalfopristin, dapsone, clofazimine and trimethoprim.

Non-limiting examples of lincosamide antibiotics include lincomycin, clindamycin, and pirlimycin.

Non-limiting examples of ansamycin antibiotics include rifampicin.

Non-limiting examples of nitrofuran antibiotics include furazolidone, nitrofurantoin, nifurfoline, nifuroxazide, nifurquinazol, nifurtoinol, nifurzide, nitrofural, ranbezolid, furaltadone, furazidine, nifuratel and nifurtimox.

Non-limiting examples of nitroimidazole antibiotics include metronidazole, tinidazole, nimorazole, dimetridazole, pretomanid, ornidazole, megazol, azanidazole, benznidazole.

Non-limiting examples of anti-cancer antibiotics include geldanamycin, herbimycin, bleomycin.

In some cases one would want to encapsulate in microparticles drugs that are non-soluble or poorly soluble in aqueous environment, and therefore are not suitable for encapsulation using the most common techniques known in the art such as W/O/W double emulsion. Poor water solubility will result in low encapsulation efficiencies. For example lipophilic drug molecules such as sterols and steroids, e.g. the anti-inflammatory drug hydrocortisone are non-soluble in water. Benzocaine is a local anesthetic drug with very low water solubility. Acidic or basic drugs in their free acid or free base form are poorly soluble, for example ciprofloxacin in its free base form is insoluble in water, while the hydrochloride salt is soluble In this case the microparticles will be prepared in alternative methods known to the skilled person in the art, such as oil/water emulsion, oil/oil emulsion, solid/oil-water technique, spray drying and more.

The drug content is optionally between 5-50% of the microparticle weight; alternatively, the polymer content is between 50-95% of the microparticle weight. Preferably the drug content is between 5-30%, and more preferably between 5-15%, of the microparticle weight.

### Additional Hydrogel Embodiments

In another embodiment it is possible to add osteoconductive materials to the gelatin matrix, in order to induce the formation of newly formed bone by facilitating cell infiltration, matrix deposition, and cell attachment in the cavity that will eventually replace the hydrogel. Example 4 shows an analysis of the mechanical properties of crosslinked gelatin matrices that include hydroxyapatite (HA) compared to control matrix without HA. The addition of HA did not inhibit the crosslinking of gelatin by transglutaminase, however it changed the mechanical properties of the gelatin matrix and made it more elastic.

In another embodiment, the crosslinked gelatin matrix containing the MP with the antibiotics is used for prophylactic purposes, for example in orthopedic reconstructive surgeries where internal fixation devices are used (e.g. plates, rods, nails, screws) or in a total knee/hip replacement. These surgeries carry a high risk of contamination, therefore applying an antibiotic eluting gelatin hydrogel at areas that are at high risk for infection (e.g. the interface of the implant and the bone, rough surfaces etc. that are prone to biofilm colonization)

In another embodiment the antibiotic eluting gelatin hydrogel can be placed on in and around hernia meshes, which are susceptible to contamination, and round the anchoring sutures or tacks used to fixate the hernia mesh to the tissue.

Drug eluting gelatin hydrogels can be sprayed on the outer surface of implants to provide a controlled release of the relevant drug. For example, crosslinked gelatin hydrogel can be used to coat vascular stent and to release anti proliferative agents such as paclitaxel.

Antibiotics eluting gelatin gels can be used also for treatment and prophylaxis of soft tissue. e.g. diabetic foot ulcers, aortic and skin grafts.

In yet another embodiment the antibiotic eluting gelatin hydrogel can be made as a dry formulation and used as a film or foam. This form has the advantage that the microparticles containing the drug are already embedded inside the cross-linkable gelatin matrix, so the reconstitution step by resuspension can be avoided. For foamed dry formulations the MPs can be integrated during the manufacturing of the dry formulation or after the drying step. During the manufacturing process, the MPs can be added either to the gelatin solution or to the enzyme solution or to the wet foam, followed by freeze drying of the wet foam. Alternatively, the NPs can be sprayed or sprinkled on the already dry foam. The MPs would adhere to the foam surface by electrostatic or Van der Waals forces. Alternatively, mixing the MPs with a volatile non aqueous solvent, that does not dissolve the polymeric MPs, allows one to spray slurry of the MPs on the external surface of the foam, where the MPs will remain attached to that surface by virtue of capillary forces. Integration of the drug eluting MPs in a film is a straightforward process where the MN are mixed with the gelatin matrix while at a liquid form, casted into a suitable mold and allowed to dry.

Drug eluting dry film or foam can be used as a bandage for treating burns (e.g. eluting antibiotics), for wound healing, as anti-inflammatory or anti-fibrosis treatment (e.g. eluting NSAID), as a hemostat (e.g. eluting clotting factors) etc.

Antibiotic eluting hydrogel can be used for soft tissue repair. For example, for treating an infected diabetic foot ulcers (DFI), especially irregular shaped tunneling foot ulcers, an injectable matrix has an advantage over a sponge or sheet form device as explained above. The matrix will be injected to fill the tunneling wound so as to maximize the contact area between the wound walls and the matrix, in order to facilitate the diffusion of the drug from the matrix into the infected wound bed. At the same time the gelatin matrix will serve as scaffold for tissue regeneration. This is based on the similarity of gelatin to collagen, which is the main constituent of the extra cellular matrix. The other constituent are GAGs, which may be mimicked by adding polysaccharides such as chitosan or hyaluronic acid.

### EXAMPLES

### Example 1

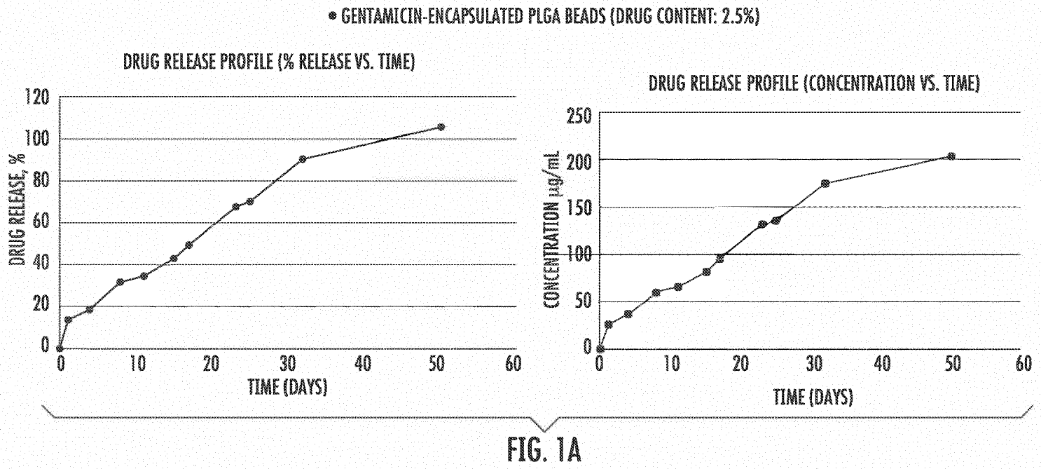

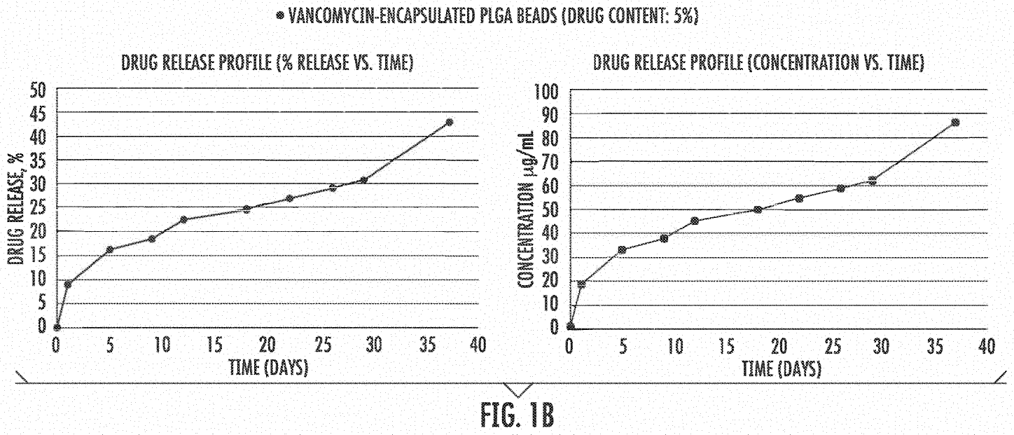

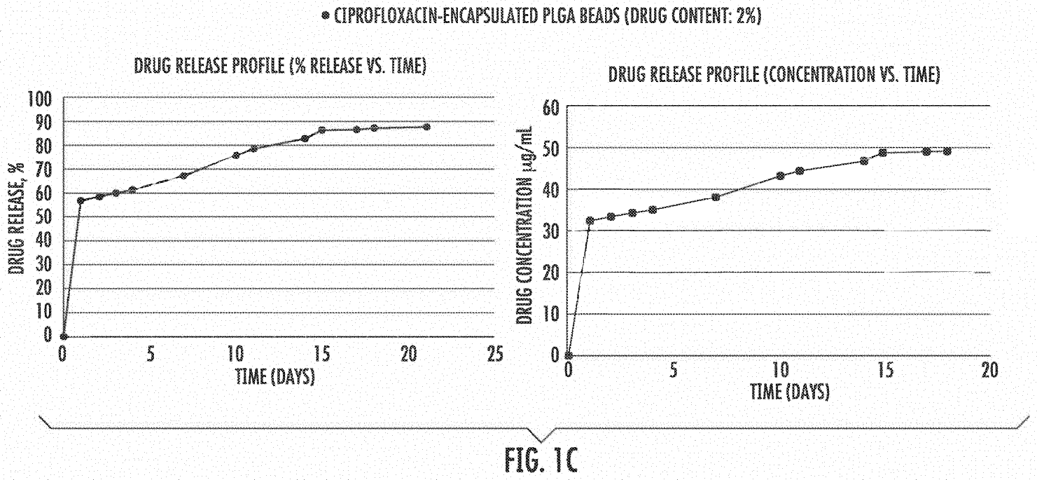

Example 1 shows in vitro release of antibiotic drugs [gentamycin (FIG. 1a), vancomycin (FIG. 1b) and ciprofloxacin (FIG. 1c)] from microparticles into PBS buffer. As shown, initially there was a burst release followed by a slower and constant release rate that followed zero order kinetics from 21 days up to at least 30 days.

PLGA (50:50) polymers, Resomer RG 503 H were purchased from Evonik Industries. Ciprofloxacin HCl, vancomycin HCl, gentamicin sulfate salt, polyvinyl alcohol (PVA, MW˜31,000), dichloromethane (DCM), paraffin oil, acetonitrile (ACN). Span 80, hexane, monobasic sodium phosphate dihydrate, NaOH, ninhydrin. PBS. Mueller Hinton broth and LB agar were purchased from Sigma Aldrich. All the materials were used as received.

Preparation of Antibiotic-Encapsulated PLGA Beads

Vancomycin/Ciprofloxacin-Encapsulated PLGA Beads

Vancomycin/ciprofloxacin-encapsulated PLGA beads were prepared by a double emulsion water-in-oil-in-oil (W/O1/O2) solvent evaporation technique. Briefly, 25 mg ciprofloxacin or 50 mg vancomycin were dissolved in 1 mL water (W) and 500 mg PLGA were dissolved in 5 mL DCM:ACN (1:1) mixture (O1). After pouring the W-phase into the O1-phase, emulsification was performed for 1 min using vortex. The first W/O1 emulsion was progressively dispersed into 100 mL of paraffin oil containing 1% Span 80 (O2) using a 10 mL syringe and a 21G needle. During the addition, emulsification was performed using a magnetic stirrer, this W/O1/O2 emulsion was stirred overnight to allow complete solvent evaporation and microsphere hardening.

The solid microspheres were recovered by filtration through a paper filter (Whatman No 1), washed three times with hexane and three times with distilled water to remove non-encapsulated drug. The microspheres were dried under vacuum at 35° C. overnight.

Gentamicin-Encapsulated PLGA Beads

Gentamicin-encapsulated PLGA beads were prepared by a double emulsion water-In-oil-in-water (W1/O/W2) solvent evaporation technique. Briefly, 25 mg gentamicin were dissolved in 250 microliter water (W1) and 500 mg PLGA were dissolved in 5 mL DCM (O). After pouring the W1-phase into the O-phase, emulsification was performed for 1 min using vortex. The first W1/O emulsion was progressively dispersed into 100 mL of a 1% (w-v) aqueous solution of PVA (W2) using a 10 mL syringe and a 21G needle. During the addition, emulsification was performed using an Ultra-Turrax homogenizer (T-18, IKA). This W1/O/W2 emulsion was stirred overnight to allow complete solvent evaporation and microsphere hardening. The solid microspheres were collected by centrifugation at 10,000 g for 10 min, and washed three times with distilled water to remove non-encapsulated drug. The microspheres were dried under vacuum at 35° C. overnight.

Drug Content and Encapsulation Efficiency

The amount of antibiotic was determined by dissolving 20 mg beads in 1 mL NaOH 1 M at 37° C. After complete dissolution, 1 mL of HCl 1 M was added to neutralize the pH. The ciprofloxacin and vancomycin concentrations were determined using spectrophotometer at 275 and 280 nm, respectively. A mixture of NaOH 1 M and HCl 1 M (1:1) was used as a blank.

The gentamicin concentration was determined by a colormetric assay: 0.5 mL of the gentamicin solution was mixed with 0.35 mL of sodium phosphate buffer (50 mM, pH 7.4) and 0.15 mL of 1.25% ninhydrin solution. The reaction occurred at 95° C. for 15 minutes and the tubes were then cooled in an ice-water bath for 10 min. The UV-visible spectra over the wavelength range of 200-700 nm were measured using the mixture of ninhydrin and the respective buffer solution at the appropriate concentrations as the blanks. The gentamicin concentration was calculated at the maximal absorbance (λmax˜315 nm).

The drug content and the encapsulation efficiency were calculated as follows:

Drug content (%)=(Drug concentration in NaOH:HCl mixture [mg/mL]*2 mL)/(Mass of beads [mg])*100

Theoretical drug content (%)=(Initial drug mass)/(Initial polymer mass)*100

Encapsulation efficiency (%)=(Actual drug content)/(theoretical drug content)*100

Microsphere Size Analysis

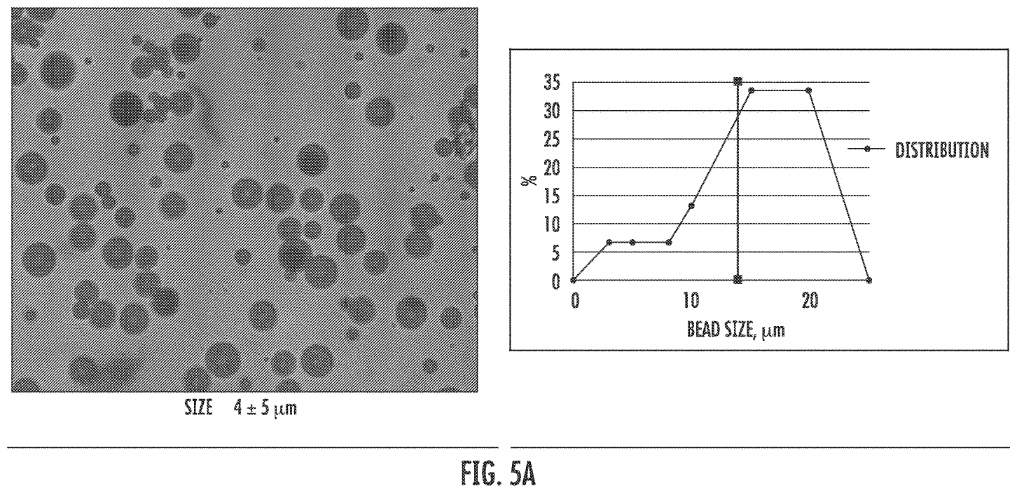

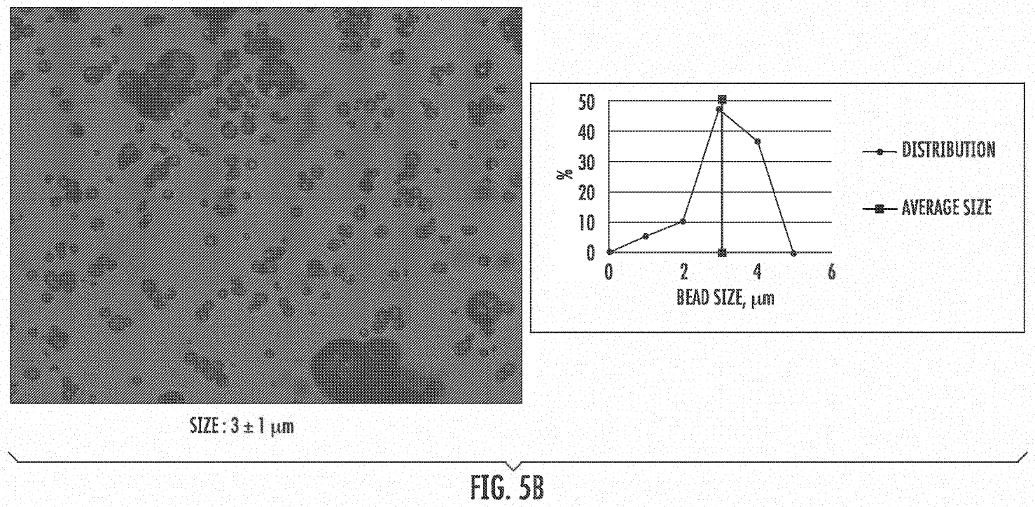

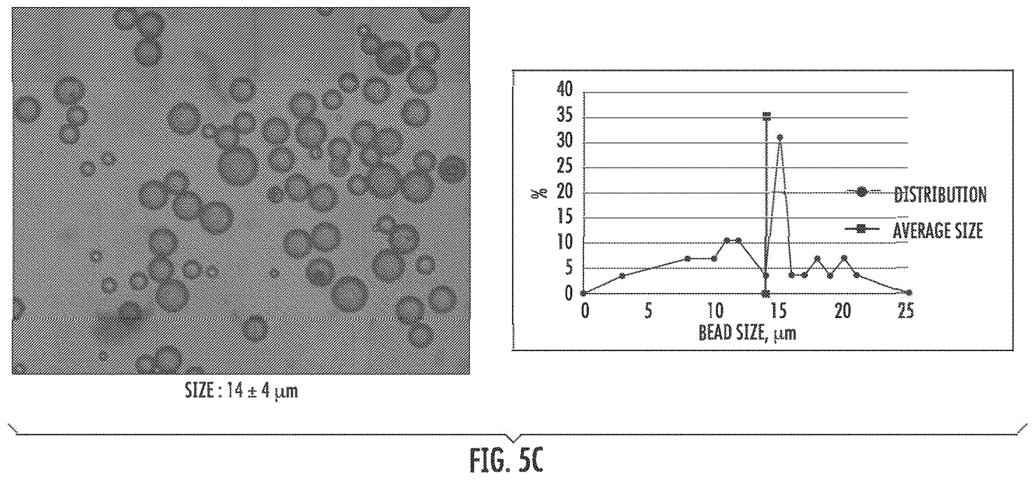

The bead size distribution of the drug-encapsulated PLGA microspheres was investigated using a microscope: Each objective of the microscope was previously calibrated using a glass slide containing a ruler of 1 mm divided into 10 μm-intervals (the microscope and accessories are from Delta-Pix Company), the average bead diameters were calculated by manually measuring the diameters of at least 20 beads from different regions of microscope pictures. The average size of each preparation of beads is shown in FIG. 5.

FIG. 5A shows, in the left panel: light microscope image of PLGA microparticles containing ciprofloxacin: and in the right panel: size distribution of the microparticles. FIG. 5B shows, in the left panel: light microscope image of PLGA microparticles containing vancomycin; and in the right panel: size distribution of the microparticles. FIG. 5C shows, in the left panel: light microscope image of PLGA microparticles containing gentamycin; and in the right panel, size distribution of the microparticles

In Vitro Drug Release Studies

The microspheres (25 mg of ciprofloxacin-encapsulated PLGA beads, 80 mg of gentamicin encapsulated PLGA beads, 40 mg of vancomycin-encapsulated PLGA beads) were placed into glass vials filled with 10 mL solution (PBS for vancomycin and ciprofloxacin-encapsulated PLGA beads and sodium phosphate buffer for gentamicin encapsulated PLGA beads). The vials were placed in an orbital shaker incubator at 37° C., where they were shaken at 120 rpm. For ciprofloxacin and vancomycin, once a day, 1.5 mL of the suspension was centrifuged and 1 mL of the supernatant was taken to spectrophotometer to measure the drug concentration. Finally, the 1.5 mL of suspension were returned back into the glass vials. For gentamicin, the reaction with ninhydrin is irreversible. 0.5 ml, of the solution was replaced each day with fresh sodium phosphate buffer to maintain a constant volume.

### Example 2

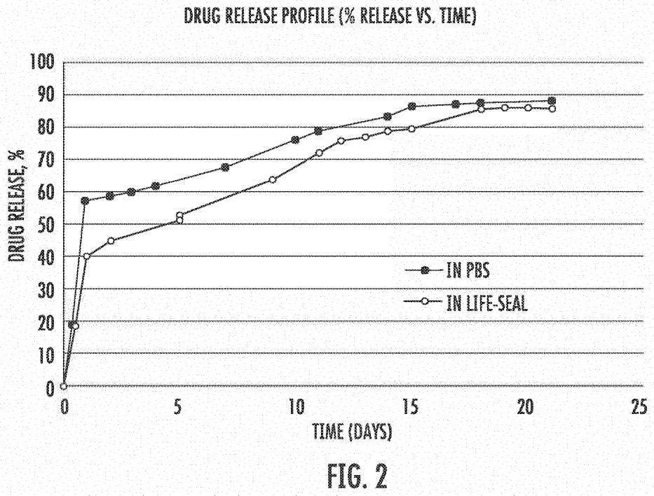

Example 2 shows release of ciprofloxacin from PLGA microparticles embedded in enzymatically crosslinked gelatin matrix. The release of the drug is somewhat slower when the MPs were embedded in gelatin matrix compared to free MPs (FIG. 2), this may be explained by the additional diffusion that is required from the drug inside the gelatin matrix after it has eluted from the NPs. Entrapment of microparticles in enzymatically crosslinked gelatin hydrogel was performed as follows.

160 mg of ciprofloxacin-encapsulated PLGA beads were added to 2.7 gr of enzyme solution. This solution was mixed with 5.0 gr of gelatin solution. 0.25 gr of the mixture was cast in a glass vial, and curing occurred at 37° C. for 15 min. 5 mL of PBS was added to the vial to wash the gel. An additional 5 mL of PBS was added and the vial was placed in an orbital shaker incubator at 37° C. where it was shaken at 120 rpm. Once a day, 1.5 mL of the cured gel extract was centrifuged and 1 mL of the supernatant was taken to spectrophotometer to measure the drug concentration. After measurement, the 1.5 mL extract was returned back into the glass vials, crosslinked gelatin without beads was casted according to the same procedure and the extract was used as blank.

### Example 3

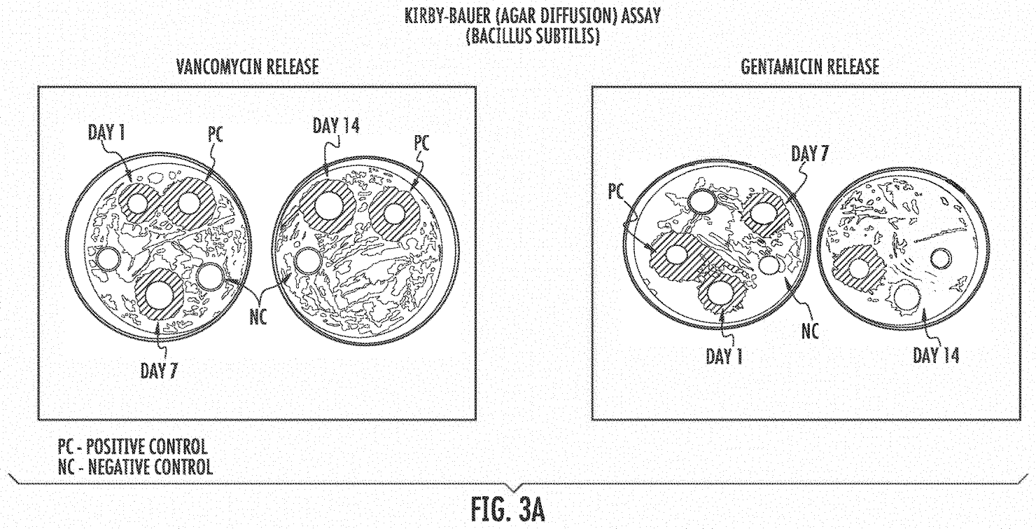

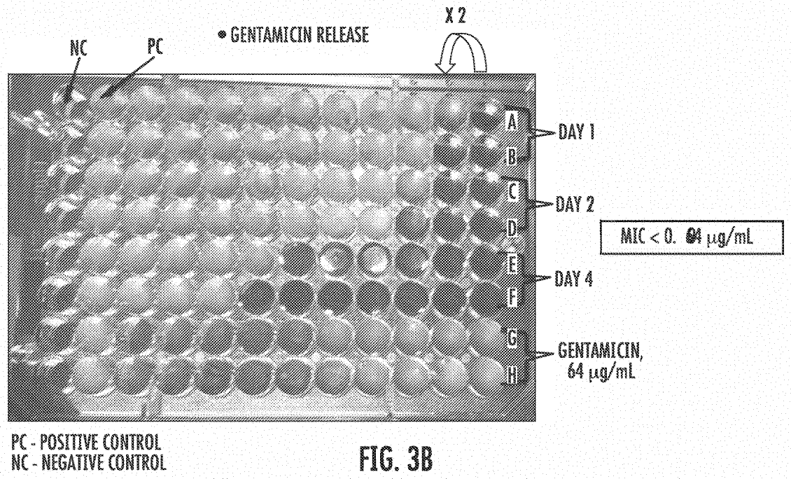

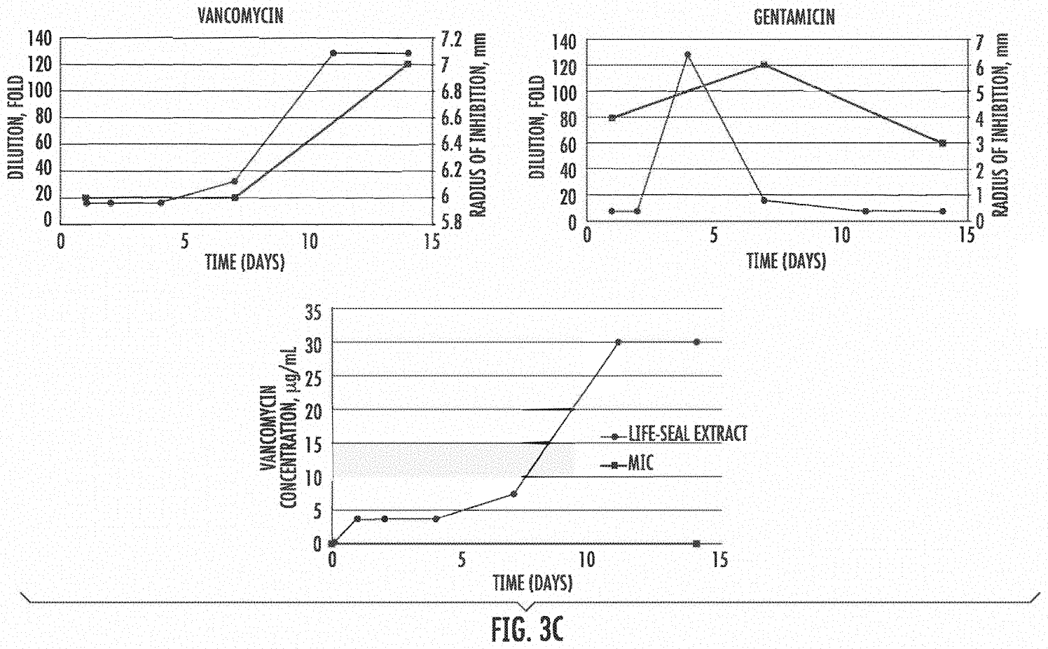

Example 3 shows the anti-microbial activity of crosslinked gelatin hydrogel containing MPs with either gentamycin or vancomycin entrapped within the MPs. The bacteria used was Bacillus subtilis, which serves as a model microorganism for gram positive bacteria. Gels that were incubated in saline for 14 days still had enough drug remaining within the matrix to induce bacteria killing, as can be seen from the ring around the gel, in agar diffusion (Kirby-Bauer) assay (FIG. 3A) or the concentration of the eluted antibiotic drug which was considerably above MIC throughout the study (FIG. 3B). The data is summarized in FIG. 3C.

Antibacterial Activity Against Bacillus subtilis (ATCC 6633, Microbiologics #0486)

6 discs of 0.2 gr crosslinked gelatin containing 2% vancomycin/gentamicin-encapsulated PLGA beads were casted in plastic mold of 12 mm diameter. After 15 mm of curing at 37° C., the gels were separately placed in glass vial filled with 1.5 mL sodium phosphate buffer. The vials were placed in an incubator at 37° C. After 1, 2, 4, 7, 11 and 14 days, the gel was taken out and the hydrogel extract was frozen until test.

The antibacterial activity of the hydrogel discs extracts was studied by employing a microdilution method (FIG. 3b). Plates were prepared under sterile conditions. 100 μL of test materials were pipetted into the first column of the sterile 96-well plate. To all other wells 50 μL of saline was added. Serial dilutions were performed using a multichannel pipette. Tips were discarded after use such that each well had 50 μL of the test material in serially descending concentrations. Then, 50 μL of Muller Hinton (NIH) broth was added to each well followed by 100 μl of bacterial suspension (prepared by growing bacteria in MH broth until OD₆₀₀ of 0.1, then diluted 100× fold in fresh MH).

Each plate had a set of controls: a column with all the solutions with the exception of the bacterial solution adding 50 μL of nutrient broth instead, and a column without antibiotic. The two last rows were used for the determination of the MIC: a gentamicin or vancomycin solution with a concentration of 64 μg/mL and 60 μg/mL, respectively, was added to the first wells and serial dilutions were performed. The plates were prepared in duplicate, and placed overnight in an orbital shaker incubator at 37° C. where a horizontal shake was performed at 120 rpm.

After 24 hours, the OD of each well was measured at 600 nm to determinate the presence or absence of bacterium. The lowest concentration at which opaque color was detected (OD>0.25) was taken as the MIC value.

Agar Disk-Diffusion Method (FIG. 3a)

3 discs of 0.2 gr crosslinked gelatin containing 2% vancomycin-gentamicin-encapsulated PLGA beads were casted as previously reported 3 additional discs without beads were also casted and used as negative control. Filters containing 30 μg antibiotics (gentamicin or vancomycin) were used as positive controls.

The gel discs with and without beads and the filter containing antibiotics were placed in LB agar plates on which 100 μl of bacterium suspension had been evenly spread. The petri dishes were placed in an orbital shaker incubator at 37° C. where a horizontal shake was performed at 120 rpm.

### Example 4

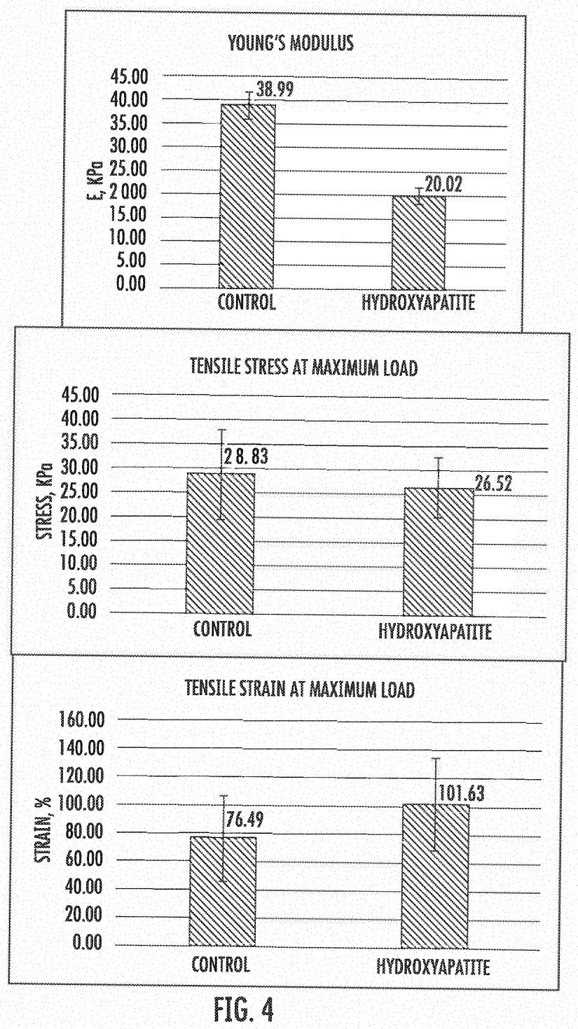

Example 4 shows mechanical testing of enzymatically crosslinked gelatin hydrogels. Hydroxyapatite was added to the gel, a control group was tested without hydroxyapatite (HA). The gels were analyzed using an Instron texture analyzer, and the tensile stress and strain at break was determined for each group (FIG. 4). The results show that the in presence of HA the crosslinked gel became more elastic, as is demonstrated by the reduced Young's modulus and increased strain at break.

Preparation of a 16% Gelatin Containing 16% Hydroxyapatite

Materials: Gelatin Type A (Gelita) Tween 20, microbial transglutaminase solution 50 U/mL, hydroxyapatite particles 5 microns in size (Sigma Aldrich).

Procedure

1.2 gr TWEEN 20 was diluted in 10 mL water. The solution was stirred for a few minutes. That solution was added to 284.3 g water and 57 g gelatin during heating and stirring until complete dissolution was achieved.

323 mg of hydroxyapatite (10% of the gelatin mass) was added to 20 g of the precedent gelatin-tween solution. The final concentrations of gelatin, hydroxyapatite and tween 20 are 16%, 16% and 0.33%, respectively

An additional solution was prepared without hydroxyapatite and was used as control

8 dog bone shaped gels from each solution were casted in Teflon coated molds. They were placed in an incubator at 37° C. for 30 min. and then transferred to a dish plate with 20 mL saline for 24 hours.

Tensile stress-strain tests were conducted (Instron 3345) at 0.5 mm/sec, at room temperature on swollen samples (FIG. 4). The measurements were carried out until the gels were torn. The tensile Young's modulus, E, was determined from the linear slope, at 10 to 30% elongation, of the tensile stress-strain curve.

### REFERENCES

### Non Patent Literature

Garvin, K. L. et al., “Polylactide/polyglycolide antibiotic implants in the treatment of osteomyelitis. A canine model” Circulation. The Journal of Bone and Joint Surgery, vol. 76, No. 10, pp 1500-1506, 1994.
Kara, 2014, Fibrin sealant as a carrier for sustained delivery of antibiotics, Journal of Clinical and Experimental Investigations 5, 194-199.
Tredwell S., Use of fibrin sealants for the localized, controlled release of cefazolin, Can J Surg. Vol. 49, No. 5, October 2006
Cashman J D. The use of tissue sealants to delver antibiotics to an orthopaedic surgical site with a titanium implant, J Orthop Sci. 2013 January; 18(1):165-74;
Foox M. In vitro microbial inhibition, bonding strength, and cellular response to novel gelatin-alginate antibiotic releasing soft tissue adhesives. Polym Adv Technol 2014; 25:516-24
Margolis J et al. Incidence of diabetic foot ulcer and lower extremity amputation among Medicare beneficiaries, 2006 to 2008, Agency for healthcare Research and Quality.

### Patents

US 20110038946 Release of antibiotic from injectable, biodegradable polyurethane scaffolds for enhanced bone fracture healing.
WO2014196943 Gel systems containing vancomycin microspheres for controlled drug release and serratiopeptidase
Penn-Barwell et al, Local Antibiotic Delivery by a Bioabsorbable Gel Is Superior to PMMA Bead Depot in Reducing infection in an Open Fracture Model. J Orthop Trauma Volume 28, Number 6, June 2014
Bennett-Guerrero E, Pappas T N, Koltun W A, et al Gentamicin-collagen sponge for infection prophylaxis in colorectal surgery. N Engl J Med. 2010:363.1038-1049
Bansal et al, MICROSPHERE. METHODS OF PREPARATION AND APPLICATIONS, A COMPARATIVE STUDY, (2011) International Journal of Pharmaceutical Sciences Review and Research, 10: 60-78
Hoare T R, Hydrogels in drug delivery: Progress and challenge, Polymer, Volume 49, Issue 8, 15 Apr. 2008, P. 1993-2007
Innocoll Announces Top-Line Data From Phase 3 Trials With COGENZIA, Inncol New Release. Nov. 3, 2016, www.innocoll.com
Edwards J et al., Debridement of diabetic foot ulcers, Cochrane Database Syst Rev. 2010 Jan. 20
Marston W A et al. Initial report of the use of an injectable porcine collagen-derived matrix to stimulate healing of diabetic foot wounds in humans, WOUND REP REG 2005:13:243-247
Campitiello F. et al. Efficacy of a New Flowable Wound Matrix in Tunneled and Cavity Ulcers: A Preliminary Report. Wounds 2015:27(6):152-157
Lipsky B A et al, Topical Application of a Gentamicin-Collagen Sponge Combined with Systemic Antibiotic Therapy for the Treatment of Diabetic Foot Infections of Moderate Severity A Randomized, Controlled, Multicenter Clinical Trial. Journal of the American Podiatric Medical Association

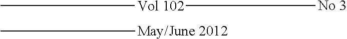

Collatamp G web site: https://collatampa.ca/Healthcare_Professionals/About_Collatamp_G
Lipsky B A et al, Topical antimicrobial agents for preventing and treating foot infections in people with diabetes. Cochrane Database of Systematic Reviews 2014. Issue 3

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A composition, comprising a hydrogel matrix and microparticles within said matrix, wherein said matrix is a freeze-dried foam, wherein said matrix comprises a cross-linkable protein and a cross-linking agent, wherein said cross-linking agent is able to cross-link said cross-linkable protein; drug; wherein said cross-linkable protein comprises gelatin and wherein said cross-linking agent comprises transglutaminase; wherein said cross-linking of said cross-linkable protein causes said cross-linkable protein to become fixated onto a tissue or anatomically defined space.

2. (canceled)

3. The composition of claim 57, wherein said drug is released from the microparticles at an average rate of release of under 5% per day.

4.-7. (canceled)

8. The composition of claim 1, wherein said cross-linking agent cross-links said cross-linkable protein only in situ.

9. The composition of claim 1, wherein said gelatin is made from type A porcine skin, bovine or fish gelatin.

10. The composition of claim 9, wherein said gelatin has a bloom of 100-300.

11.-13. (canceled)

14. The composition of claim 1, wherein said transglutaminase is microbial.

15. The composition of claim 1, wherein said microparticles comprise a biodegradable polymer selected from the group consisting of: an aliphatic polymer, a polycarbonate polymer and a polyamino acid polymer.

16.-18. (canceled)

19. The composition of claim 15, wherein the biodegradable polymer comprises a homopolymer.

20.-22. (canceled)

23. The composition of claim 57, wherein said drug comprises one or more antibiotics, analgesic drugs, anti-inflammatory drugs, and/or anti-tumor drugs.

24.-40. (canceled)

41. The composition of claim 57, wherein the composition comprises a combination of drugs.

42.-45. (canceled)

46. The composition of claim 1, wherein a polymer content of said particles is between 50-95% of the microparticle weight.

47. The composition of claim 1, wherein a size range of said microparticles is 0.5-50 microns.

48. (canceled)

49. The composition of claim 1, wherein said microparticles are dispersed in the protein component, the cross-linking agent component or both.

50. The composition of claim 49, wherein an amount of microparticles in each component ranges between 10 mg/ml and 80 mg/ml.

51. The composition of claim 49, wherein an amount of microparticles in the final formulation following the mixing of said components ranges between 10 mg/ml and 80 mg/ml.

52.-53. (canceled)

54. The composition of claim 57, wherein drug elution time from the microparticles is adjusted so that the drug elutes from the microparticles over the course of 2 to 6 weeks.

55. The composition of claim 15, wherein the biodegradable polymer comprises a copolymer of two or more monomers.

56. The composition of claim 15, wherein the biodegradable polymer comprises mixture of polymers.

57. The composition of claim 1, wherein said microparticles comprise one or more drugs.

58. The composition of claim 57, wherein the one or more drugs comprise minocycline and/or rifampicin.

59. A hernia mesh comprising the composition of claim 1, wherein the composition is placed on, in, and/or around the hernia mesh.
